(19) United States
(12) Patent Application Publication
(10) Pub. No.: US 2014/0080923 A1
(43) Pub. Date: Mar. 20, 2014

(54) STEVIA BASED SWEETENING COMPOSITION

(71) Applicant: Herman Weiss, Rishon LeZion (IL)

(72) Inventor: Herman Weiss, Rishon LeZion (IL)

(73) Assignee: STEVINOL LTD., Tel-Aviv (US)

(21) Appl. No.: 14/116,034

(22) PCT Filed: May 8, 2012
(86) PCT No.: PCT/IL2012/050169

§ 371 Date: Nov. 6, 2013

Related U.S. Application Data
(60) Provisional application No. 61/483,755, filed on May 9, 2011.

(51) Int. Cl. A23L 1/236; A61K 8/60

(52) U.S. Cl. CPC A23L 1/2363; A61K 8/602
USPC 514/777; 426/548; 426/72; 141/26

(57) ABSTRACT

A liquid composition that includes at least one steviol glycoside dissolved in glycerol, and a method for preparation thereof, preferably at a temperature above 100° C. The steviol glycoside is extracted from Stevia rebaudiana plant leaves. The steviol glycoside may include rebaudioside A, rebaudioside B, rebaudioside C, rebaudioside D, rebaudioside E, rebaudioside F, dulcoside A, steviol, stevioside, and/or steviolbioside. The relative concentration by weight of the rebaudioside A may be at least approximately 95%. The relative concentration by weight of the glycerol may be at least 70%. The liquid composition may further include additional ingredients, such as vitamins, minerals, extracts, preservatives, and/or other food additives. A dropper may be used for extracting individual drops of the liquid composition, such as into a beverage. The liquid composition may be used as a food additive, a sweetener, and/or a dietary supplement.

### FIELD OF THE DISCLOSED TECHNIQUE

The disclosed technique generally relates to natural food additives.

### BACKGROUND OF THE DISCLOSED TECHNIQUE

The Stevia genus is a plant belonging to the Asteraceae (i.e., daisy or sunflower) family, and is native to subtropical and tropical regions in South America, such as Paraguay and Brazil. The genus includes about 240 different species, one of which—Stevia rebaudiana (often colloquially referred to as “stevia”)—is characterized by considerable sweetness. The source of this sweetness is a group of chemical compounds known as glycosides, primarily stevioside and rebaudioside A, which are nearly 300 times sweeter than sucrose. The aglycone (non-sugar part) of the glycoside is a diterpene called steviol, and so these chemical compounds in stevia are more specifically referred to using the term “steviol glycosides”. Additional trace amounts of other steviol glycosides are also present in the stevia plant, including: rebaudioside B, rebaudioside C, rebaudioside D, rebaudioside E, and dulcoside A.

Stevia has virtually no calories or carbohydrates, is heat stable, and is non-fermentative, and thus has garnered considerable attention globally as an appealing alternative to sugar. Since stevia has a negligible effect on blood glucose levels, it is particularly attractive to be used as a sweetener for diabetics or those on a reduced-carbohydrate diet. In addition, stevia is considered non-addictive and leaves a negligible aftertaste. Indigenous South American tribespeople have been using stevia to sweeten drinks for centuries. In Japan, stevia has been used as a natural sweetener and food additive since the early 1970s. Nowadays, stevia is cultivated and used as a food additive in many countries worldwide, especially in South America and the Far East, although it was banned for consumption in the United States in the early 1990s unless labeled as a “dietary supplement” (and so could not legally be termed a “sweetener”). However in 2008, the U.S. Food and Drug Administration (FDA) approved the use of reubaudioside-A as a food additive. As a result, sweeteners based on steviol glycosides have been available in the U.S. under various consumer brand names, such as: Rebiana and Truvia (both Cargill, Inc. brands), PureVia (PepsiCo, Inc.), and Enliten (Corn Products International, Inc.). In Europe, stevia has been imported and consumed since the 1980s, but was categorized as a “novel food” by the European Commission (EC) in 1997, and subsequently banned as a food additive by the EC Scientific Committee for Food in 2001.

Issues of safety and health repercussions surrounding the consumption of stevia are subject to a certain degree of controversy and debate in scientific circles. Although stevia is generally not considered carcinogenic, some research studies have reported identifying steviol as a mutagen in laboratory rats. Other studies have had mixed results with respect to the toxicology and possible adverse health effects of stevia constituents.

Besides its usage as a sweetener, stevia extracts have been utilized in a variety of additional applications, ranging from stimulating alertness levels, facilitating digestive functioning, and treating skin blemishes. Stevia extracts are widely used in various herbal medicines, and are present in some topical creams and lotions. Stevia has been found to inhibit the growth of some bacteria responsible for tooth decay, leading to its inclusion in certain toothpastes in China. It is believed that stevia may help heal various ailments, such as high uric acid levels, hypertension, heartburn, high blood sugar, and obesity. Some studies have found that the ingestion of stevia by chickens results in reduced egg breakage. Other studies have found that stevia consumption diminishes desire for tobacco or alcohol.

Typically, extracts based on steviol glycosides, such as the now FDA-approved rebaudioside A, are diluted within another liquid. For example, the rebaudioside A extract is administered into a beverage intended for consumption, in order to sweeten the beverage. However, a solution made up of water and a rebaudioside A extract, such as one consisting of approximately 90% water and 10% rebaudioside A extract, is unstable and the rebaudioside A extract tends to settle as a residue, constituting a deficiency when dissolving such an extract within a water-based liquid.

U.S. Patent Application No. 2007/0082103 to Magomet et al., entitled “Process for manufacturing a sweetener and use thereof”, is directed to a process for producing a highly purified sweetener from the extract of the Stevia rebaudiana Bertoni plant for use in various food products and beverages. The process involves drying Stevia rebaudiana plant leaves, treating the leaves with water to obtain an extract of mixed sweet glycosides, and filtering the extract. The filtrate is treated with a base such as calcium hydroxide, and then treated with trivalent iron chloride, followed by desalting, decolorizing and evaporating the filtrate until dryness, to obtain purified sweet glycosides. Purified rebaudioside A and stevioside can be isolated therefrom via alcohol precipitation and ultrafiltration.

U.S. Patent Application No. 2008/0226795 to May et al., entitled “Non-nutritive sweetened beverages with glycerine”, is directed to a beverage product containing at least one non-nutritive sweetener, in an amount sufficient to provide perceptible sweetening. The beverage product further contains glycerine, in an amount sufficient to enhance mouthfeel without a detectable change in taste, e.g., in an amount between about 0.01 wt. % and 7.0 wt. % of the finished beverage. The non-nutritive sweetener may be at least one steviol glycoside. The beverage may include other natural ingredients (e.g., juices, fruit flavors, natural acids, botanical flavors, spices) and other suitable ingredients (e.g., flavorings, acids, edible salts, carbonation, colorants).

PCT Patent Application Publication No. WO 2009/137838 to Cargill, Incorporated, entitled “Sweetener, methods of preparing sweetener and applications thereof”, is directed to a high intensity sweetener and a taste modifying composition. The composition includes at least one congruent flavor volatile (e.g., vanillin) and/or at least one non-congruent flavor volatile (e.g., alpha ionone). The high intensity sweetener may be a steviol glycoside, such as rebaudioside A. The sweetener may further contain a bulking material, and may be used to create a food, beverage or pharmaceutical product, such as a tabletop sweetener.

Japanese Patent No. JP59139309 entitled “Emulsified cosmetic”, is directed to an emulsified cosmetic, such as a cream or lotion, that incorporates rebaudioside A with glycerol monoester. The cosmetic includes 0.01-5.0 wt. %, and preferably 0.02-2.0 wt. %, rebaudioside A, that acts as an emulsifying agent. The glycerol monoester enhances the emulsifying ability and increases stability over time. The cosmetic may further incorporate a natural surface active substance, such as xanthan gum, to add emulsifying stability to an oily substance.

### SUMMARY OF THE DISCLOSED TECHNIQUE

In accordance with one aspect of the disclosed technique, there is thus provided a composition including at least one steviol glycoside and glycerol (C₃H₅(OH)₃). The steviol glycoside may be extracted from Stevia rebaudiana plant leaves. The steviol glycoside may include at least rebaudioside A. The relative concentration by weight of the rebaudioside A may be at least approximately 95%. The steviol glycoside may include at least another compound including steviol, stevioside, steviolbioside, rebaudioside B, rebaudioside C, rebaudioside D, rebaudioside E, rebaudioside F, or dulcoside A. The composition may further include at least one ingredient including a vitamin, a mineral, an extract, a preservative, a food additive. A product which includes the composition may be a food additive, a sweetener, a dietary supplement, a beverage, or a cosmetic product.

The composition may be a liquid composition of at least one steviol glycoside dissolved in glycerol. The relative concentration by weight of the glycerol in the liquid composition may be above 30%, or 70%, or about 93%. A dropper for extracting individual drops of the liquid composition may be applied.

The composition may be a granulate of at least one steviol glycoside granulated with glycerol. The granulate may be a powder. The relative concentration by weight of the glycerol in the granulate may be above 0.5%, under 30% and above 1%, or under 3% and above 2%.

According to another aspect of the disclosed technique, there is provided a method of preparation of a composition, including obtaining an extract including at least one steviol glycoside, and mixing the extract with glycerol (C₃H₅(OH)₃). The steviol glycoside may be extracted from Stevia rebaudiana plant leaves. The steviol glycoside may include at least rebaudioside A. The relative concentration by weight of the rebaudioside-A may be at least 95%. The composition may be used as a food additive, a sweetener, or a dietary supplement, administered into a beverage, or included in a cosmetic product.

Mixing may include dissolving the extract in glycerol to obtain liquid composition. The relative concentration by weight of the glycerol in the liquid composition may be above 30%, or 70%, or about 93%. Dissolving may be performed at a temperature above 100° C., between 120 ° C.-170 ° C., or between 140 ° C.-160 ° C.

Mixing may include granulating the extract with glycerol to obtain a granulate. The relative concentration by weight of the glycerol in the granulate may be above 0.5%, above 1% and below 30%, or above 2% and below 3%.

### DETAILED DESCRIPTION OF THE EMBODIMENTS

The disclosed technique overcomes the disadvantages of the prior art by providing a steviol glycoside based composition. The composition is preferably a liquid composition that dissolves in a stable manner within a liquid solution. The composition is applicable to be administered into a beverage, such as via individual drops, for sweetening the beverage prior to consumption, as an alternative to sugar and sugar substitutes or other artificial sweeteners or flavorings. Alternatively, the composition is a granulate or a powder.

In accordance with the disclosed technique, Stevia rebaudiana plant leaves are treated to obtain an extract that includes at least one steviol glycoside, using purification and extraction techniques known in the art. The steviol glycoside may include rebaudioside A. The rebaudioside A may be present in a relative concentration (by weight) of at least approximately 95% (e.g., 95-99%), at which the extract would currently be considered approved by the U.S. Food and Drug Administration (FDA) for use as a “food additive”. Alternatively, rebaudioside A may be present in the extract at a lower relative concentration (e.g., above 10%). The extract may include additional steviol glycosides besides rebaudioside A, such as: steviol, stevioside, steviolbioside, rebaudioside B, rebaudioside C, rebaudioside D, rebaudioside E, rebaudioside F, and dulcoside-A. The extract may also include additional chemical compounds present in the Stevia rebaudiana plant. When preparing a liquid composition, the extract is then dissolved in glycerol (C₃H₅(OH)₃), preferably at a temperature above 100° C. At this dissolving temperature, the solution remains stable such that there is substantially no residue settling over time in the solution. Dissolving is preferably performed at temperatures ranging between 100° C.-200° C. Further preferably the dissolving temperature is between 120° C.-170° C., at which the solution liquefies and becomes transparent (but may subsequently turn white). Further preferably dissolving takes place at temperatures between 140° C.-160° C. At 160° C. dissolving was found to be faster than at 140° C. However, the dissolving may generally be implemented at temperatures up to about 290° C., at which the boiling point of the glycerol is reached. The relative concentration (by weight) of glycerol in the solution is preferably above 30% and further preferably above 70%. For example, the solution includes about 7% of the extract dissolved in about 93% glycerol.

The resultant composition is applicable for use as a food additive, and in particular as a sweetener, owing to the sweetness properties of the steviol glycosides and the glycerol within the composition. The liquid composition of the disclosed technique is particularly suitable to be added into a beverage, in the form of individual drops administered via a dropper, such as an eyedropper, or other suitable mechanism. The precise amount of the composition to be added can be controlled by the individual to match his/her particular preferences (i.e., depending on the desired degree of sweetness in the final beverage product), such as by administering a selected number of drops of predefined quantity using a measurement calibrated dropper. Exemplary beverage products may include: coffee, tea, soft drinks, carbonated beverages, juices, milk, wine, alcoholic beverages (particularly wine and beer), energy drinks, water, combinations thereof, and the like. Alternatively, the composition of the disclosed technique may be added to a food product for sweetening, such as confectionary items or baked goods. Such beverage or food products may include other additives and/or sweeteners in addition to the composition of the disclosed technique. For example, the composition of the disclosed technique may be administered as a food additive together with other ingredients, (e.g., vitamins, minerals, extracts, preservatives, and the like), such as in the form of a mixture or combined solution. However, since glycerol functions as a food preservative and inhibits the growth of micro-organisms (e.g., bacteria, fungi) that lead to spoilage, it is generally unnecessary to include additional preservatives in the composition of the disclosed technique. It is noted that the beverage may be provided with a selected amount of the composition as part of the initial process of preparing and producing the beverage, e.g., prior to being packaged and commercially distributed to the consumer, such as at the beverage production facility. Alternatively the composition may be supplied directly to the end consumer, who then applies the desired amount of the composition into the beverage before consumption (i.e., using an ‘eyedropper’ or other suitable mechanism as outlined hereinabove). The beverage may be at any suitable temperature when the composition is added therein (i.e., the beverage may be exceedingly hot or exceedingly cold), provided that the beverage remains primarily in liquid form.

It is appreciated that the composition of the disclosed technique is entirely in a liquid form after its preparation, and remains as such. The composition is stable and fully dissolves into any type of beverage in which it is administered into, including both water-based liquids and oil-based liquids, without leaving any visible residue. Furthermore, the composition includes various beneficial properties inherent to steviol glycosides, such as: a negligible amount of calories and carbohydrates, a negligible effect on blood glucose, a non-addictive nature, a negligible aftertaste, and heat stability, while maintaining the desired sweet taste. Additionally, the composition of the disclosed technique may be marketed, distributed and sold in the United States as a “food additive” or “sweetener” when rebaudioside-A is present at a relative concentration of at least approximately 95% (following FDA approval as discussed hereinabove). In any case, the composition of the disclosed technique may still be marketed, distributed and sold in the United States as a “dietary supplement”, regardless of the rebaudioside A relative concentration.

According to another aspect of the disclosed technique there is provided a steviol glycoside based dry granular composition (or powder) dissolvable within a liquid solution. Reference herein below to granular composition, granulate composition, dry composition, granulate, powder, and the like, is synonymous, and any reference to a granulate only, or to one of such forms, is made for short and inherently references all of the other forms. The granulate is applicable to be administered into a beverage, such as via spoon-sized servings, for sweetening the beverage prior to consumption, as an alternative to sugar and sugar substitutes or other artificial sweeteners or flavorings.

In accordance with the disclosed technique, Stevia rebaudiana plant leaves are treated to obtain an extract that includes at least one steviol glycoside, using purification and extraction techniques known in the art. The steviol glycoside may include rebaudioside A. The rebaudioside A may be present in a relative concentration (by weight) of at least approximately 95% (e.g., 95-99%), at which the extract would currently be considered approved by the U.S. Food and Drug Administration (FDA) for use as a “food additive”. Alternatively, rebaudioside A may be present in the extract at a lower relative concentration (e.g., above 10%). The extract may include additional steviol glycosides besides rebaudioside A, such as: steviol, stevioside, steviolbioside, rebaudioside B, rebaudioside C, rebaudioside D, rebaudioside E, rebaudioside F, and dulcoside-A. The extract may also include additional chemical compounds present in the Stevia rebaudiana plant. The extract is then granulated with glycerol (C₃H₅(OH)₃), using dry granulation techniques known in the art, to receive a granulate (or a powder, or a dry composition) which includes both the extract and the glycerol. Alternatively, wet granulation techniques may be applied. The relative concentration (by weight) of glycerol in the granulate is above 0.5%. However, above 50% and even above 30%, it is difficult to obtain solid granulates, as the the glycerol tends to remain liquid. Preferably, the relative concentration of glycerol in the granulate is 0.5%-10%, and further preferably between 1%-3%, or 2%-3%.

The resultant granulate composition (or powder) is applicable for use as a food additive, and in particular as a sweetener, owing to the sweetness properties of the steviol glycosides and the glycerol within the granulate. The granulate (or powder) of the disclosed technique is particularly suitable to be added into a beverage, in the form of servings administered via teaspoon, sachet or other suitable mechanism. The precise amount of the granulate composition to be added can be controlled by the individual to match his/her particular preferences (i.e., depending on the desired degree of sweetness in the final beverage product), such as by administering a selected number of teaspoons of predefined quantity using a measurement calibrated teaspoon. Exemplary beverage products may include: coffee, tea, soft drinks, carbonated beverages, juices, milk, wine, alcoholic beverages (particularly wine and beer), energy drinks, water, combinations thereof, and the like. Alternatively, the granulate (or powder) of the disclosed technique may be added to a food product for sweetening, such as confectionary items or baked goods. Such beverage or food products may include other additives and/or sweeteners in addition to the granulate composition of the disclosed technique. For example, the granulate composition of the disclosed technique may be administered as a food additive together with other ingredients, (e.g., vitamins, minerals, extracts, preservatives, and the like), such as in the form of a mixture or combined solution. However, since glycerol functions as a food preservative and inhibits the growth of micro-organisms (e.g., bacteria, fungi) that lead to spoilage, it is generally unnecessary to include additional preservatives in the granulate of the disclosed technique. It is noted that the beverage may be provided with a selected amount of the granulate (or powder) as part of the initial process of preparing and producing the beverage, e.g., prior to being packaged and commercially distributed to the consumer, such as at the beverage production facility. Alternatively the granulate (or powder) may be supplied directly to the end consumer, who then applies the desired amount of the granulate composition into the beverage before consumption (i.e., using a teaspoon, sachet or other suitable mechanism as outlined hereinabove). The beverage may be at any suitable temperature when the granulate composition is added therein (i.e., the beverage may be exceedingly hot or exceedingly cold), provided that the beverage remains primarily in liquid form.

It is appreciated that the granulate (or powder) of the disclosed technique is entirely in a solid form after its preparation, and remains as such. The granulate (or powder) is stable and fully dissolves into any type of beverage in which it is administered into, including both water-based liquids and oil-based liquids, without leaving any visible residue. Furthermore, the granulate composition includes various beneficial properties inherent to steviol glycosides, such as: a negligible amount of calories and carbohydrates, a negligible effect on blood glucose, a non-addictive nature, a negligible aftertaste, and heat stability, while maintaining the desired sweet taste. Additionally, the granulate (or powder) of the disclosed technique may be marketed, distributed and sold in the United States as a “food additive” or “sweetener” when rebaudioside-A is present at a relative concentration of at least approximately 95% (following FDA approval as discussed hereinabove). In any case, the granulate composition of the disclosed technique may still be marketed, distributed and sold in the United States as a “dietary supplement”, regardless of the rebaudioside A relative concentration.

The liquid composition and/or the granulate and/or the powder of the disclosed technique may be potentially used in other applications and may be included in other types of products. For example, the liquid composition and/or the granulate and/or the powder may be included in skin creams, lotions, ointments or other forms of cosmetics or topical treatments.

It will be appreciated by persons skilled in the art that the disclosed technique is not limited to what has been particularly shown and described hereinabove.

1. A liquid composition comprising at least one steviol glycoside dissolved in glycerol (C₃H₅(OH)₃), wherein the relative concentration by weight of said glycerol is above 70%.

2. The liquid composition of claim 1, wherein said steviol glycoside is extracted from Stevia rebaudiana plant leaves.

3. The liquid composition of claim 1, wherein said steviol glycoside comprises at least rebaudioside A.

4. The liquid composition of claim 3, wherein the relative concentration by weight of said rebaudioside A is at least approximately 95%.

5. The liquid composition of claim 1, wherein said steviol glycoside comprises at least another compound selected from the list consisting of:
steviol;
stevioside;
steviolbioside;
rebaudioside B;
rebaudioside C;
rebaudioside D;
rebaudioside E;
rebaudioside F;
dulcoside A; and
any combination of the above.

6. The liquid composition of claim 1, further comprising at least one ingredient selected from the list consisting of:
a vitamin;
a mineral;
an extract;
a preservative; and
a food additive.

7. A product comprising the liquid composition of claim 1, wherein said product is selected from the list consisting of:
a food additive;
a sweetener;
a dietary supplement;
a beverage; and
a cosmetic product.

8-10. (canceled)

11. The liquid composition of claim 1, wherein the relative concentration by weight of said glycerol is about 93%.

12. A dropper for extracting individual drops of the liquid composition of claim 1.

13-17. (canceled)

18. A method of preparation of a liquid composition, the method comprising the procedures of:
obtaining an extract comprising at least one steviol glycoside; and
dissolving said extract in glycerol (C₃H₅(OH)₃), wherein the relative concentration by weight of said glycerol is above 70%.

19. The method of claim 18, wherein said steviol glycoside is extracted from Stevia rebaudiana plant leaves.

20. The method of claim 18, wherein said steviol glycoside comprises at least rebaudioside-A.

21. The method of claim 18, wherein the relative concentration by weight of said rebaudioside-A is at least 95%.

22. The method of claim 18, wherein said liquid composition is used as a food additive, a sweetener, or a dietary supplement.

23. The method of claim 18, wherein said liquid composition is administered into a beverage.

24. The method of claim 18, wherein said liquid composition is included in a cosmetic product.

25-27. (canceled)

28. The method of claim 18, wherein the relative concentration by weight of said glycerol is about 93%.

29. A method of preparation of a liquid composition, the method comprising the procedures of:
obtaining an extract comprising at least one steviol glycoside; and
dissolving said extract in glycerol (C₃H₅OH)₃) at a temperature above 100° C.

30. The method of preparation of claim 29, wherein said temperature is between 120° C.-170° C.

31. The method of preparation of claim 30, wherein said temperature is between 140° C.-160° C.

32-35. (canceled)
